(19) United States
(12) Patent Application Publication
(10) Pub. No.: US 2022/0105263 A1
(43) Pub. Date: Apr. 7, 2022

(54) SYSTEM AND METHODS FOR DELIVERING TESTOSTERONE REPLACEMENT DRUG THERAPIES

(71) Applicant: Channa Amarasekera, Chicago, IL (US)

(72) Inventor: Channa Amarasekera, Chicago, IL (US)

(21) Appl. No.: 17/062,890

(22) Filed: Oct. 5, 2020

(51) Int. Cl. A61M 5/142; A61K 31/568; A61M 5/162; A61M 5/168

(52) U.S. Cl. CPC A61M 5/14276; A61M 2005/14252; A61M 5/162; A61M 5/16809; A61M 5/1684; A61M 5/14248; A61M 2205/18; A61M 2205/587; A61M 2205/3327; A61M 2205/3553; A61M 2205/581; A61M 2205/3334; A61M 2205/50; A61M 2005/14284; A61K 31/568

(57) ABSTRACT

The present disclosure relates generally to the administration of testosterone replacement therapy, and more particularly to a system and methods for monitoring and analyzing testosterone levels of a patient for dynamically controlling and/or managing delivery of testosterone. The system may include an implantable medical device including a pumping mechanism operatively coupled to an analyte sensor. The analyte sensor may collect a patient's testosterone levels and communicate that information to the pumping mechanism. Based on the information collected by the analyte sensor, the pumping mechanism may deliver one or more testosterone replacement drug therapies to the patient. Advantageously, the system may automatically regulate the delivery of said one or more testosterone replacement drug therapies based on a testosterone level of the patient, while minimizing patient oversight of the administration process and reducing discomfort.

### FIELD OF THE DISCLOSURE

The present disclosure relates generally to the administration of testosterone as it relates to testosterone replacement therapy, and more particularly to a system and methods for monitoring and analyzing testosterone levels of a patient for dynamically controlling and/or managing the delivery of testosterone.

### BACKGROUND

Hypogonadism in men is commonly characterized by low levels of the male sex hormone testosterone. The hypogonadal condition is sometimes linked with a number of physiological conditions, such as diminished interest in sex, impotence, reduced lean body mass, decreased bone density, depressed mood, and reduced energy levels. Also, testosterone deficiency has been linked to a number of health problems, including osteoporosis, cardiovascular disease, diabetes and metabolic syndrome, erectile dysfunction and libido diminution, depression, fatigue, and anemia.

It is estimated that 10-30% of men worldwide are hypogonadal, with higher levels in older men and men with chronic medical conditions. With increasing awareness of the negative impacts of testosterone deficiency, annual prescription testosterone sales in the U.S. have increased from $18 million in 1988 to over $2 billion in 2013.

Treatment for the hypogonadal condition may include the use of testosterone replacement drug therapies. Various methods of delivering testosterone have been considered and developed to provide suitable replacement therapies. Examples include testosterone gels and patches, intranasal gels, buccal patches, intramuscular injections, subcutaneous pellets, and a new oral twice daily pill formulation.

However, the use of conventional methods for testosterone replacement is a time-consuming process. Patients often need to frequently administer suitable replacement therapies through use of patches, gels, tablets, and/or other forms. Those with low or no testosterone due to certain medical conditions may need frequent visits to healthcare providers for injections, if they are unable to inject themselves. Methods for delivering testosterone that may take less time and reduce distracting patients from their daily activities would provide a variety of advantages.

Also, many conventional methods for testosterone replacement do not facilitate dynamically targeting treatment levels. Specifically, conventional methods often provide for no diurnal pattern control and certain delivery methods, such as injections, peak testosterone levels that overshoot a desired range with marked declines in levels over a short period of time. This may be detrimental to a patient because testosterone levels fluctuate in undesirable ways throughout the day.

Additionally, the administration of testosterone replacement drug therapy may be difficult to stop through use of conventional methods. For example, the effects of certain injections of drug therapies last for a number of weeks, testosterone undecanoate injections' effects last for two to three months, and the effects of implantable pellets may last for three to six months. In cases where patients have adverse reactions to the medication, such as hypertension and/or irritability, a controlled administration of drug therapies that may be halted quickly and easily would be beneficial.

Other disadvantages associated with conventional methods for delivery of testosterone replacement include patient discomfort. For example, with frequent injections, patients may experience pain and/or may experience irritation at the application site. Also, the use of a gel for testosterone replacement may result in unwanted transfer of testosterone, which may be worrisome for women and children. In addition, with oral pills, patients also may need to consume fatty meals for better absorption. If testosterone levels with replacement are higher than desired, it can result in high levels of dihydrotestosterone (DHT), which can shrink hair follicles, causing hair to grow out looking thinner and more brittle, as well as fall out faster. Uncontrolled levels of testosterone can also lead to polycythemia, as well as devastating cardiac and vascular complications such as heart attacks and strokes.

Therefore, there is a need for a system and methods for monitoring and analyzing testosterone levels of a patient and dynamically controlling and/or managing the delivery of testosterone replacement that reduces discomfort and the frequency with which patients or physicians must administer the medication. The present invention satisfies this long-felt need.

### SUMMARY

The present disclosure relates to a system and methods for monitoring and analyzing testosterone levels of a patient for dynamically controlling and/or managing delivery of testosterone for testosterone replacement therapy. Advantageously, the system may automatically regulate the delivery of said one or more testosterone replacement drug therapies based on a testosterone level of the patient, while minimizing patient oversight of the administration process and reducing discomfort. Moreover, the system solves other various technical problems associated with prior systems for dispensing testosterone replacement drug therapies.

An aspect of the present disclosure is an implanted medical device for delivering one or more testosterone replacement drug therapies. The medical device may include a front surface including one or more openings for one or more channels. At least one reservoir may connect to the one or more channels, each reservoir configured to hold a testosterone replacement drug therapy. A catheter assembly may extend from a side surface of the medical device and connect to at least one reservoir for delivering testosterone to the patient. A pumping mechanism may connect to the catheter and be configured to dynamically regulate the delivery of the one or more testosterone replacement drug therapies based on a testosterone level of the patient.

Each opening of the medical device may include a pierceable and elastically reclosable membrane for receiving a needle. The channels may include a detector for detecting the presence of the needle when a reservoir is being refilled. In addition, each reservoir may include a sensor configured to measure the amount of testosterone present within the reservoir such that a user is notified when refilling is necessary. The front surface of the medical device may further include light emitting diodes positioned around each opening. The light emitting diodes may illuminate in response to a refilling operation or a notification.

In certain embodiments of the medical device, the pumping mechanism may be operatively coupled to a continuous analyte sensor configured to measure the testosterone level of the patient. The analyte sensor may extend from a side surface of the medical device opposite the catheter. Alternatively, the analyte sensor may be remotely implanted subcutaneously within the patient.

A transceiver of the medical device may be configured to receive a control signal from one or more external device. In response to the control signal, the pumping mechanism may be configured to increase, decrease or stop the delivery of the one or more testosterone replacement drug therapies. Examples of testosterone replacement drug therapies may include unmodified testosterone, testosterone propionate, testosterone enanthate, testosterone undecanoate, testosterone cypionate, testosterone undecylentate, other testosterone derivatives, human chorionic growth hormone, conjugated estrogens, estradiol, esterified estrogens, progesterone, methylprogesterone, progesterone derivates, and anastrazole.

Another aspect of the present disclosure is a wearable device including an analyte sensor, a transceiver, and a processor. The processor is operatively coupled to the analyte sensor and transceiver. The processor is further operative to obtain testosterone levels collected using the analyte sensor and send that data to a second device over a wireless link using the transceiver.

The wearable device may further include a needle having one or more carbon nanotubes, such as single-walled carbon nanotubes or multi-walled carbon nanotubes. Through use of the carbon nanotubes, the analyte sensor of the wearable device is configured to collect testosterone using one or more methods including, for example, a corona phase molecular recognition technique, an anti-testosterone antibody, and an aptamer specific for testosterone.

Another aspect of the present disclosure is a system for dynamically controlling delivery of a testosterone replacement. The system may include an analyte sensor and an implantable medical device having a pump. The medical device may further include a transceiver, a processor operatively coupled to the transceiver, and a memory storing one or more modules with instructions that the processor may execute.

The processor of the system may analyze testosterone levels collected by the analyte sensor and determine a flow rate for dispensing one or more testosterone replacement drug therapies. The processor may then output a control signal to the pump causing the pump to deliver the testosterone replacement drug therapies to a patient at the determined flow rate.

In certain embodiments, the processor is further operative to obtain the flow rate from a look up table of the memory. The lookup table may include a plurality of entries, each entry corresponding to the one or more testosterone replacement drug therapies. In addition, the processor may receive from one or more external devices, the flow rate and adjust the control signal to, for example, increase, decrease or stop the delivery of testosterone replacement.

Also, the medical device of the system may include one or more sensors operatively coupled to the processor. The processor may analyze information collected by the one or more sensors and communicate one or more notifications to the patient based on the collected information.

While the invention is susceptible to various modifications and alternative forms, specific exemplary embodiments thereof have been shown by way of example in the drawings and have herein been described in detail. It should be understood, however, that there is no intent to limit the invention to the particular embodiments disclosed, but on the contrary, the intention is to cover all modifications, equivalents, and alternatives falling within the scope of the invention as defined by the appended claims.

### BRIEF DESCRIPTION OF THE DRAWINGS

FIG. 1A is a perspective front view of an exemplary implantable medical device for delivering testosterone;

FIG. 1B is a perspective back view of the exemplary implantable medical device of FIG. 1A;

FIG. 2 is a perspective side view of an exemplary implantable medical device according to one embodiment;

FIG. 3 is a perspective side view of an exemplary implantable medical device according to one embodiment;

FIG. 4 is a top view of an exemplary implantable medical device according to one embodiment;

FIG. 5A illustrates an analyte sensor physically connected to a medical device implanted within a patient;

FIG. 5B illustrates an analyte sensor connected via a physical wire to a medical device implanted within a patient;

FIG. 5C illustrates an analyte sensor positioned remotely and operatively connected to a medical device implanted within a patient;

FIG. 6 illustrates an exemplary belt including a charger for charging a medical device implanted within a patient;

FIG. 7 illustrates a block diagram of an exemplary medical device;

FIG. 8 illustrates an exemplary implantable analyte sensor;

FIG. 9 illustrates an exemplary non-implantable wearable device including an analyte sensor;

FIG. 10A illustrates a method for detecting levels of testosterone within a patient including the use of an aptamer;

FIG. 10B illustrates a method for detecting levels of testosterone within a patient using a Corona Phase Molecular Recognition (CoPhMoRE) technique; and

FIG. 10C illustrates a method for detecting levels of testosterone within a patient using an anti-testosterone antibody.

### DETAILED DESCRIPTION

The present disclosure relates generally to the administration of testosterone, and more particularly to a system and methods for monitoring and analyzing testosterone levels of a patient for dynamically controlling and/or managing delivery of testosterone replacement.

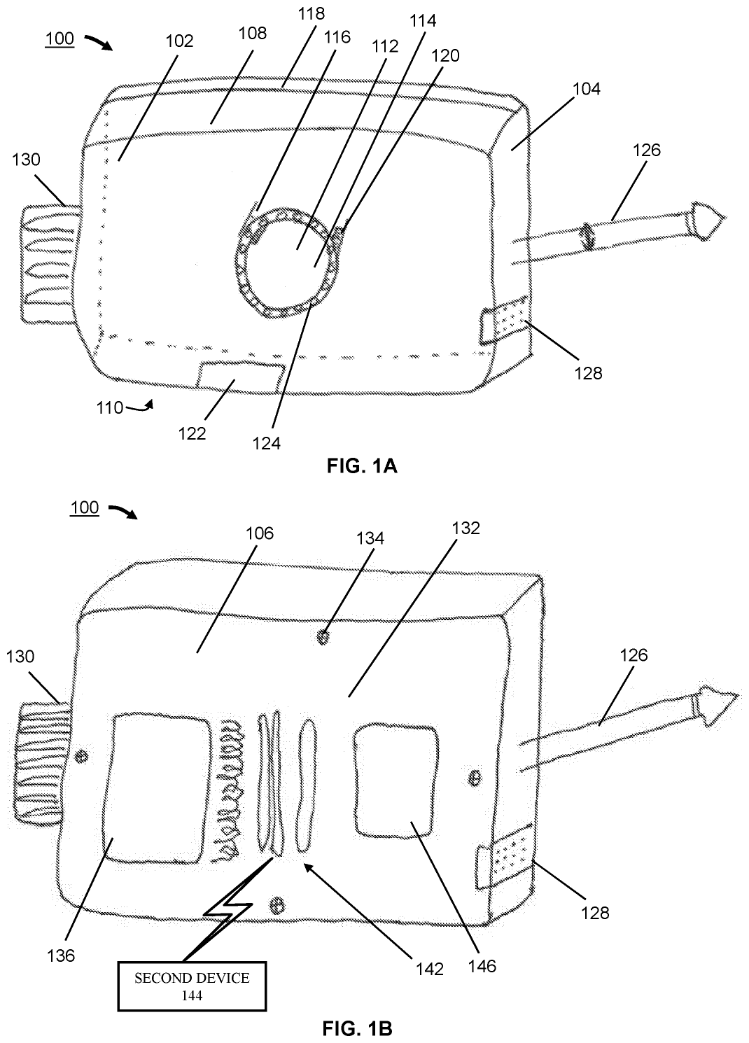

FIG. 1A and FIG. 1B illustrate an exemplary medical device 100 for delivering one or more testosterone replacement drug therapies to a patient. As shown, the medical device 100 includes a front surface 102, side surfaces 104, a back surface 106, a top surface 108, and a bottom surface 110.

One or more portions of the medical device 100 may be constructed from metals and/or alloys, such as stainless steel, cobalt-chrome alloy, titanium, and nickel-titanium alloy. In some embodiments, the components of medical device 100 are made from materials safe for use with magnetic resonance imaging (MRI). While medical device 100 is shown as substantially rectangular shaped, other shapes are contemplated including, for example, square, round, and oval.

Medical device 100 may have dimensions ranging from about three centimeters by two centimeters by one centimeter to about five centimeters by four centimeters by two centimeters, and preferably be about four centimeters by three centimeters by two centimeters. In one embodiment, the dimensions of medical device 100 are four and half centimeters by three and a half centimeters by one and half centimeters. It is contemplated that medical device 100 may be shrunk consistent with manufacturing techniques and materials.

As shown in FIG. 1A, front surface 102 may include an opening 112. Opening 112 may have a circumference ranging from about one to about five centimeters, and preferably be about one to about three centimeters. In one embodiment, opening 112 has a circumference of about two centimeters.

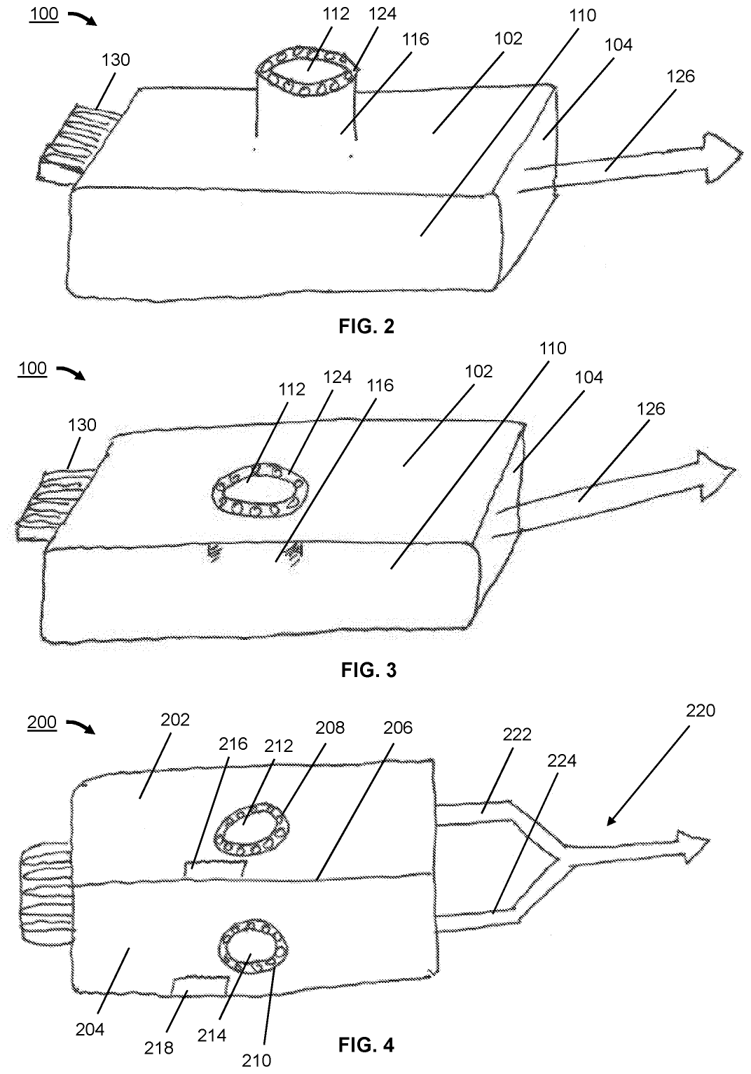

Opening 112 may include a pierceable and elastically reclosable membrane 114, such as a semi-translucent silicone rubber membrane. Membrane 114 may open to a channel 116 for receiving a component, such as a needle, capable of refilling a reservoir 118. As shown in FIG. 2, channel 116 may extend away from front surface 102 such that opening 112 is raised approximately one and a half centimeters above the front surface 102. Alternatively, as shown in FIG. 3, opening 112 is flush against front surface 102 of medical device 100.

Channel 116 may be in fluid communication with the reservoir 118, which is structured to hold testosterone. Examples of forms of testosterone or other medications that may be stored in reservoir 118 include unmodified testosterone, testosterone propionate, testosterone enanthate, testosterone undecanoate, testosterone cypionate, testosterone undecylenate, other testosterone derivatives, human chorionic growth hormone, conjugated estrogens, estradiol, esterified estrogens, progesterone, methylprogesterone, other progesterone derivates, and anastrazole.

During a refill operation, a needle may pierce the membrane 114 of the opening 112, travel through channel 116, and thereby gain access to the reservoir 118.

Components of medical device 100 may further include one or more sensors. For example, channel 116 may include a detector 120 for detecting the presence of the needle. In addition, the reservoir 118 may include a drug therapy sensor 122 configured to determine the amount of testosterone replacement within the reservoir 118. Other sensors, such as pressure sensors, flow sensors, and/or valves may be integrated into the channel 116 and/or the reservoir 118 to facilitate monitoring of the flow rate and/or pressure during the refilling process and controlling pump operation, as detailed below.

Referring back to FIG. 1A, a visualization ring 124 may surround the opening 112 on the front surface 102 of the medical device 100. Visualization ring 124 may be visible through a layer of skin so as to visually indicate a position of the opening 112 to a user (such as a physician) after the medical device 100 is implanted within a patient's body. For example, medical device 100 may be implanted in the subcutaneous tissue in the upper, outer quadrant of either buttock, the subcutaneous tissue of the abdomen, hip, thigh, arm or any area of the patient's body below the neck with sufficient amounts of adipose tissue. In certain embodiments, medical device 100 may be placed within a range of about one to four centimeters under the skin, and preferably about three centimeters under the skin.

Visualization ring 124 may include one or more light emitting diodes (LEDs) that illuminate during refilling and/or indicate a status of the medical device 100. In another embodiment, the visualization ring 124 may move and/or vibrate in a manner that gives the user a tactile sensation confirming the location of the opening 108. Other methods for visualizing opening 108 are contemplated.

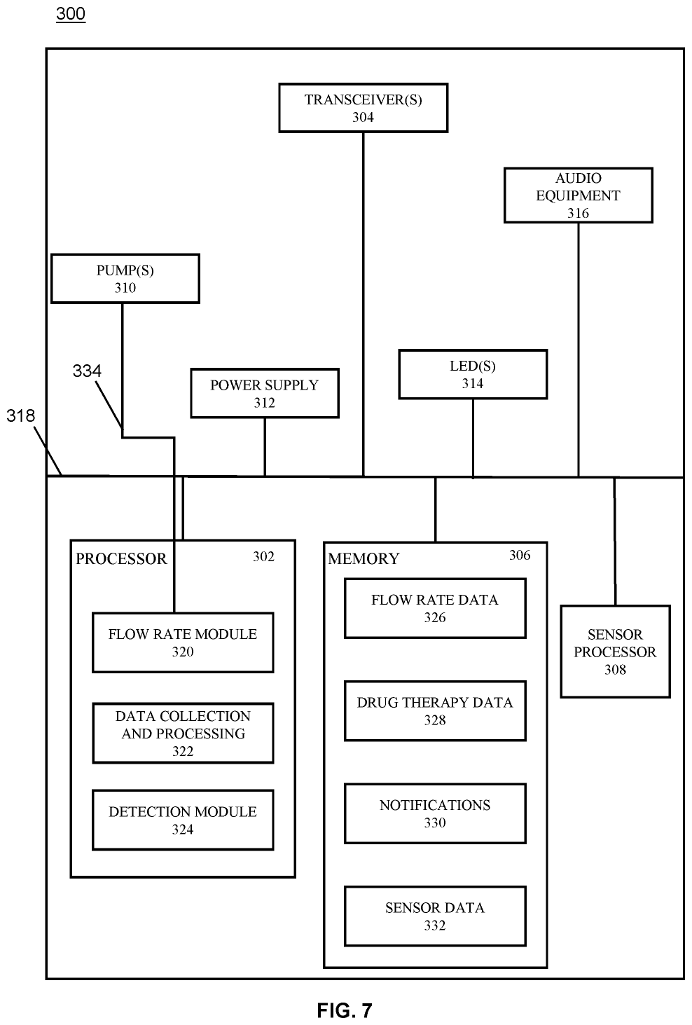

Medical device 100 of FIGS. 1A and 1B may further include a tubular drug dispensing cannula 126 extending from a side surface 104. One or more pumping mechanisms (see FIG. 7) may be fluidly coupled to the reservoir 118 for creating fluidic pressure to facilitate delivery of the testosterone replacement to the patient via cannula 126. The length of cannula 126 may range from about five centimeters to about ten centimeters, and preferably be about eight centimeters in length. The diameter of cannula 126 may range from about three millimeters to about seven millimeters, and preferably have a diameter of about five millimeters.

Side surface 104 may further include a speaker 128 for communicating notifications and/or alerts to a patient. Speaker 128 may have dimensions ranging from about three millimeters by three millimeters by one centimeter to about six millimeters by six millimeters by three centimeters, and preferably be about five millimeters by five millimeters by two centimeters.

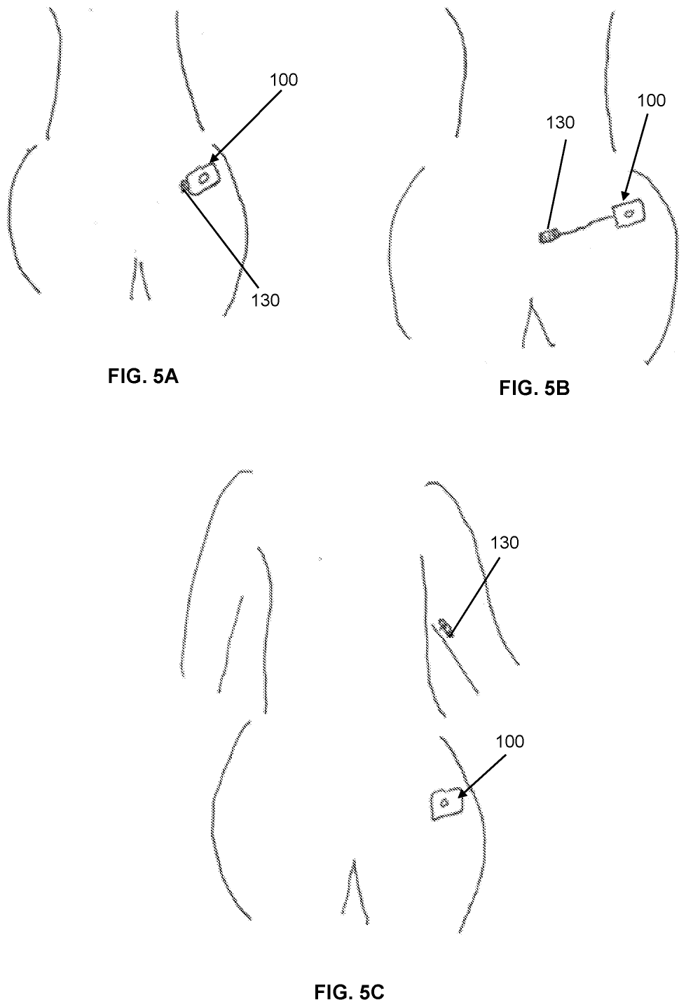

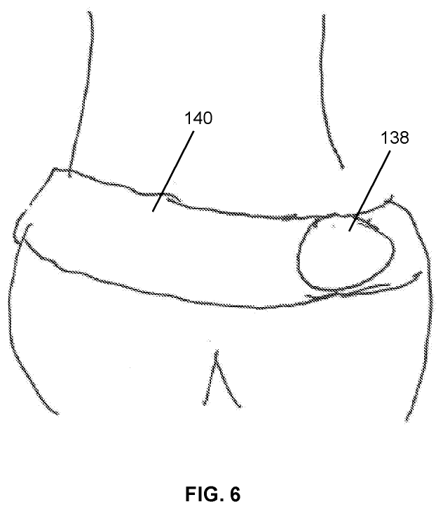

Certain embodiments of medical device 100 may include an analyte sensor 130 configured to make quantitative analyte measurements. As shown in FIG. 5A, analyte sensor 130 may extend from a side surface 104 opposite the cannula 126. In other embodiments, analyte sensor 130 is in communication with medical device 100 via a physical wire. See FIG. 5B. In yet another embodiment, analyte sensor 130 may be positioned at a remote location and operatively connected to the medical device 100. See FIG. 5C. In other embodiments, medical device 100 receives quantitate analyte measurements from an external device, such as a mobile device or wearable device, as detailed below.

Referring back to FIG. 1B, medical device 100 may include a compartment 132 accessible by removing hardware 134, such as screws, from back surface 106. Compartment 132 may include a power system 136 for powering the various components. Power system 136 may include a power management system, one or more power sources (e.g., battery, alternating current (AC)), a recharging system, a power failure detection circuit, a power converter or inverter, and any other components associated with the generation, management, and distribution of power in implantable medical devices.

Examples of suitable power sources may include non-rechargeable lithium batteries, as well as rechargeable Li-ion, lithium polymer, thin-film (e.g., Li-PON), nickel-metal-hydride, and nickel cadmium batteries. In the case of rechargeable power sources, as shown in FIG. 6, electrical energy may be transcutaneously transmitted from a charger 138 that a patient may wear on an article of clothing, such as a belt 140. Belt 140 may be positioned on the patient such that the charger 138 aligns with power source 136 for efficient charging. Alternatively, a patient may wear spandex pants, and position the charger 138 between the skin and spandex at the location of power source 136. Other examples of suitable power sources may include a capacitor or motion-generated energy systems.

FIG. 1B further illustrates wireless circuitry 142 of medical device 100 for communicating with one or more external devices 144. The medical device 100 and the one or more external devices 144 may go through a pairing procedure or a connection procedure depending on the wireless technology employed. Examples of external devices 144 may include a mobile device such as, but not limited to, a mobile phone (also referred to as a “smartphone”), a laptop computer, personal digital assistant (PDA), or similar device. In addition, one or more external devices 144 may be microprocessor-controlled medical devices, such as a smart patch, implantable pacemakers, cardioverters, defibrillators, neural stimulators, drug-administering devices, or other implantable devices.

The various illustrative components of medical device 100 may be implemented or performed via a processor 146. Processor 146 may be a general purpose processor, a special purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, any conventional processor, controller, microcontroller, or state machine. A general purpose processor may be considered a special purpose processor while the general purpose processor is configured to execute instructions (e.g., software code) stored on a computer readable medium. A processor may also be implemented as a combination of computing devices, such as a combination of a DSP and a microprocessor, a plurality of microprocessors, one or more microprocessors in conjunction with a DSP core, or any other such configuration.

It should be appreciated that medical device 100 may have more or fewer components than shown, optionally may combine two or more components, or optionally may have a different configuration or arrangement of the components. For example, a needle including a cannula may extend from back surface 106 such that medical device 100 may be implantable or worn on a patient's skin. In another example, medical device 100 may be covered with a waterproof material and/or coating and may further include a rubberized port for refilling.

The various components shown in FIG. 1A and FIG. 1B may be implemented in hardware, software, or a combination of both hardware and software, including one or more signal processing and/or application specific integrated circuits.

FIG. 4 illustrates a top view of another exemplary medical device 200. As shown, medical device 200 may include a first reservoir 202 and a second reservoir 204, each reservoir 202, 204 configured to store a testosterone replacement drug therapy. A partition 206 may be positioned between first reservoir 202 and second reservoir 204. Each reservoir 202, 204 may be fluidly coupled to one or more pumps (FIG. 5) for creating fluidic pressure to facilitate delivery of the testosterone replacement drug therapy to the patient.

Similar to the exemplary medical device 100 of FIG. 1A, a front surface 206 of medical device 200 includes visualizing rings 208, 210 that surround openings 212, 214 to each corresponding reservoir 202, 204. Each reservoir 202, 204 may further include a drug level sensor 216, 218 configured to determine an amount of each testosterone replacement drug therapy contained within the medical device 200.

As shown in FIG. 4, medical device 200 may further includes a catheter 220. Catheter 220 may include a first lumen 222 connected to the first reservoir 202 and a second lumen 224 connected to the second reservoir 204. In one operation, testosterone replacement drug therapies dispensed through each corresponding lumen 222, 224 may coalesce during delivery to a patient.

FIG. 7 is a block diagram of an exemplary medical device 300. Medical device 300 may include at least one processor 302, one or more transceivers 304, non-volatile, non-transitory memory 306, sensor processor 308, one or more pumps 310, a power supply 312, LED(s) 314, and audio equipment 316.

One or more pumps 310 may include a mechanism that delivers one or more testosterone replacement drug therapies in some metered or other desired flow dosage to the patient from a reservoir via a catheter or cannula, as discussed above. Suitable pumps may include peristaltic pumps, solenoid pumps, diaphragm pumps, piston pumps, piezoelectric pumps, and other like known pumps.

All of the components of medical device 300 may be operatively coupled to the processor 302 by one or more internal communication buses 318. In the example embodiment illustrated in FIG. 7, the processor 302 runs the flow rate module 320, data collection and processing 322, and detection module 324. In some embodiments, the sensor processor 308 monitors and stores in memory 306 from various sensors including a gyroscope and an accelerometer (which may be separate or integrated in a single package) as well as other sensors such as, but not limited to, analyte sensors, pressure sensors, flow sensors, temperature sensors, altitude sensors, motion sensors, position sensors, and other sensors relating to the operations of medical device 300.

The memory 306 is non-volatile and non-transitory and stores executable code and instructions for various applications associated with medical device 300, such as flow rate data 326, drug therapy data 328, notifications 330, and sensor data 332. The processor 302 is operative to, among other things, launch and execute the operating data collection and processing 322 and detection module 324.

The processor 302 also runs the flow rate module 326 which is operatively connected over an interface 334 to one or more pumps 310. In certain embodiments, the processor 302 is operative to determine the flow rate based on a lookup table stored in the memory 306. The lookup table may have a plurality of entries, wherein each entry corresponds to a drug therapy and flow rate.

Flow rate module 326 may define an amount of the testosterone drug therapy to be delivered to a patient from a reservoir via a catheter or cannula. The instructions may further specify a time at which the testosterone replacement will be delivered and the time interval over which the testosterone replacement will be delivered. The amount of the testosterone replacement and the time over which the testosterone replacement is delivered may be functions of a flow rate at which fluid is delivered. In other examples, a quantity of the testosterone replacement may be delivered according to one or more physiological characteristics of a patient, such as the patient's circadian testosterone cycle.

Based on flow rate data 326, drug therapy data 328, and sensor data 332, processor 302, operatively coupled to flow rate module 320, is operative to send a controlled signal to one or more pumps 310. In particular, processor 302 executes flow rate module 320 which instructs one or more pumps 310 to increase, decrease or stop the delivery of said one or more testosterone drug therapies.

In certain embodiments, one or more pumps 310 may be set to infuse at a set rate, which may be set by a physician, dispensing a standard dose of testosterone replacement every hour. For example, the one or more pumps 310 may infuse 0.1 mg of testosterone replacement per hour continuously. Alternatively, the one or more pumps 310 may infuse at a set level or range of testosterone replacement. For example, the one or more pumps 310 may be set to deliver testosterone such that a patient's testosterone levels are between 500 nanograms (ng) per decilitre (dL)-600 ng/dL, and would adjust the level of testosterone replacement drug infused based on testosterone levels within the patient. Also, the parameters for the one or more pumps 310 may to release enough testosterone replacement drug therapies to achieve physiologic serum levels of testosterone, such as ranging from about 300 ng/dL to about 1000 ng/dL, and preferably be about 400 ng/dL to about 600 ng/dL. In one embodiment, one or more pumps may release testosterone replacement drug therapies to achieve testosterone levels within a patient of about 500 ng/dL.

In certain embodiments, one or more pumps 310 may be configured to deliver testosterone ranging from about 0.1 mg to about 0.7 mg daily, and preferably ranging from about 0.2 mg to about 0.5 mg daily. In one embodiment, one or more pumps 310 may be configured to deliver a total of 0.3 mg of testosterone daily, in 15-90 minute intervals. A 90-minute interval may mimic the Luteinizing Hormone (the hormone that drives testosterone production) pulses released by the body. For example, if 0.3 mg of testosterone is delivered per day, one or more pump 310 may release 0.019 mg of testosterone every 90 minutes.

In certain embodiments, one or more pumps 310 may stop the delivery of testosterone and send the user a notification. For example, a pump of medical device 300 may stop the delivery of testosterone to a patient if a sensor in communication with one or more pumps 310 does not detect a change or a lowering of testosterone levels within a patient. In addition, medical device 300 may communicate an alert or notification to a patient and/or physician, as this may indicate that the sensor is malfunctioning.

One or more pumps 310 may also be configured to deliver variable amounts of testosterone depending on the time of day, with goal levels being set for each hour. This may facilitate dispensing testosterone replacement in a way that closely mimics the circadian testosterone cycle, particularly in younger men. For example, the goal level for 8 AM may be set to 900 ng/dL, at 3 PM it could be 700 ng/dL, and 550 ng/dL from 8 PM to 1 AM, 750 ng/dL at 3 AM with a linear rise to 900 ng/dL at 8 AM. The values for each hour can be programmed, for example, by a health care provider using an application for managing the patient's testosterone replacement therapy, or a pre-set circadian mode may be available with the ability to specify the desired peak and nadir testosterone replacement drug levels.

In certain embodiments, medical device 300 may determine the flow rate at which one or more pumps 300 must infuse the medication to reach a certain level by infusing 0.01 mg of testosterone followed by 0.015 mg in 90 minutes, increasing the dose in 0.005 mg increments until a dose of 0.03 mg is delivered. In certain embodiments, medical device 300 may be configured to use machine learning algorithms to learn what the patient's testosterone levels did with each dose, and calculate how much the patient's testosterone level changes with a particular dose of testosterone. Medical device 300 may then use this information to achieve desired testosterone levels throughout the day.

As mentioned above, an application may be used for controlling and/or managing medical device 100 for delivering of a testosterone replacement to a patient. The application may include one or more interfaces, such as an interface for a health care provider and an interface for a patient. Medical device 300 may have a unique serial number that is linked to the patient's information. Examples of patient information include input demographic information such as name, date of birth, address, phone number, and e-mail address, and medical/clinical information, such as height, weight, blood pressure, and pulse.

Through use of the application, such as via a smart phone, a patient may access data corresponding to real time testosterone levels. The data may include values corresponding to an amount of testosterone replacement that has been infused over a specified period of time. In addition, the application may present users with one or more questionnaires on, for example, hypogonadism (qADAM), depression (CES-D), erectile function (IIEF), and benign prostatic hyperplasia (AUA symptom score).

In certain embodiments, the application may alert the patient when 6-months of therapy has elapsed, indicating a need to, for example, have blood drawn by a physician for a PSA level, complete blood count, liver function tests, a lipid panel, and/or have blood pressure checked as recommended by the FDA for men on testosterone replacement. In addition, a health care provide may use the application to indicate that the patient has performed lab testing and/or that a joint decision was made to forgo testing. In certain embodiments, the application may be used to shut off medical device 300, such as in cases of poor patient compliance. The number of months between testing can be customized by the physician.

In certain embodiments, patients, through use of the application, will have the ability to change the range of testosterone levels delivered by medical device 300. For example, if the patient feels no relief from the symptoms of hypogonadism at a goal testosterone of 400-500 ng/dL, the physician can set options for the patient to change goals to 500-600 ng/dL or 600-700 ng/dL in the absence of adverse events from testosterone replacement.

As shown in FIG. 7, the medical device 300 may include one or more wireless transceivers 304. Each of the wireless transceivers 304 may be implemented as physical wireless adapters or virtual wireless adapters. A single physical wireless adapter may be virtualized using software into multiple virtual wireless adapters. A physical wireless adapter typically connects to a hardware-based access point. A virtual wireless adapter typically connects to a software based wireless access point. For example, a virtual wireless adapter may allow ad hoc communications between peer devices such as a mobile device or wearable device. Various embodiments may use a single physical wireless adapter implemented as multiple wireless adapters, multiple physical wireless adapters, multiple physical wireless adapters each implemented as multiple virtual wireless adapters or a combination thereof.

Wireless transceivers 304 may comprise or implement various communication techniques to allow medical device 300 to communicate with a second device, such as external device 144 of FIG. 1. For example, the wireless transceivers 304 may implement various types of standard communication elements designed to be interoperable with a network, such as one or more communications interfaces, network interfaces, network interface cards, radios, wireless transceivers, wireless communication media, physical connectors etc. Examples of communications may include, cables, fiber optics, propagated signals, radio frequency, infrared, and other wireless media.

In addition, medical device 300 may implement different types of wireless transceivers 304. Each wireless transceiver may implement or utilize a same or different set of communication parameters to communicate information between the external device and/or other various devices. Examples of communication parameters may include a communication protocol, a communication standard, a radio-frequency band, a radio, a transceiver, a radio processor, an access point parameter, modulation and coding scheme, media access control layer parameter, physical layer parameter and any other communication parameter affecting operations for the wireless transceivers 304.

Wireless transceivers 304 also may implement different communication parameters offering varying bandwidths, communication speeds or transmission range. In another embodiment, the wireless transceiver 304 may comprise WLAN baseband hardware designated to communicate information over a wireless local area network (WLAN). Examples of suitable WLAN systems offering lower range data communications services may include the IEEE 802.xx series of protocols, such as the IEEE 802.11a/b/g/n series of standard protocols and variants (also referred to as “WiFi”). It may be appreciated that other wireless techniques may be implemented, and the embodiments are not limited in this context.

Although not shown, the medical device 300 may further include one or more device resources commonly used in implantable, mobile, and/or wearable devices, such as various computing and communications platform hardware and software components. Such device resources may be used in the collection of data to be used by the flow rate module 320, data collection and processing 326 or detection module 324. Some examples of device resources may include, without limiting, a co-processor, graphics processing unit, a chipset platform control hub, network interfaces, location devices, sensors (eg. proximity, pressure, biometric, thermal, environmental, etc.), portable power supplies, application programs, system programs and the like.

Memory 306 may be operatively coupled to the processor 302 via the internal communications buses 318 as shown, may be integrated with, or distributed between one or more processors, or may be some combination of operatively coupled memory and integrated memory. Memory 306 may be any suitable non-volatile, non-transitory memory that may be used to load executable instruction or program code to a processor or other device such as those that may benefit from the features described herein. Furthermore, it is to be understood that any of the above described example components in the example medical device 300, without limitation, may be implemented as software (i.e. executable instructions or executable code) or firmware (or a combination of software and firmware) executing on one or more processors, or using ASICs (application-specific-integrated-circuits), DSPs (digital signal processors), hardwired circuitry (logic circuitry), state machines, FPGAs (field programmable gate arrays) or combinations thereof. In embodiments in which one or more of these components is implemented as software, or partially in software/firmware, the executable instructions may be stored in the operatively coupled, non-volatile, non-transitory memory 306, and may be accessed by the processor 302, sensor processor 308, or other processors, as needed. The non-volatile, non-transitory memory 306 may be part of a computer program product, and is loaded into or written on the medical device 300 via a removable storage drive, hard drive, or communications interface. The software described herein need not reside on the same or a singular medium in order to perform the inventions described herein.

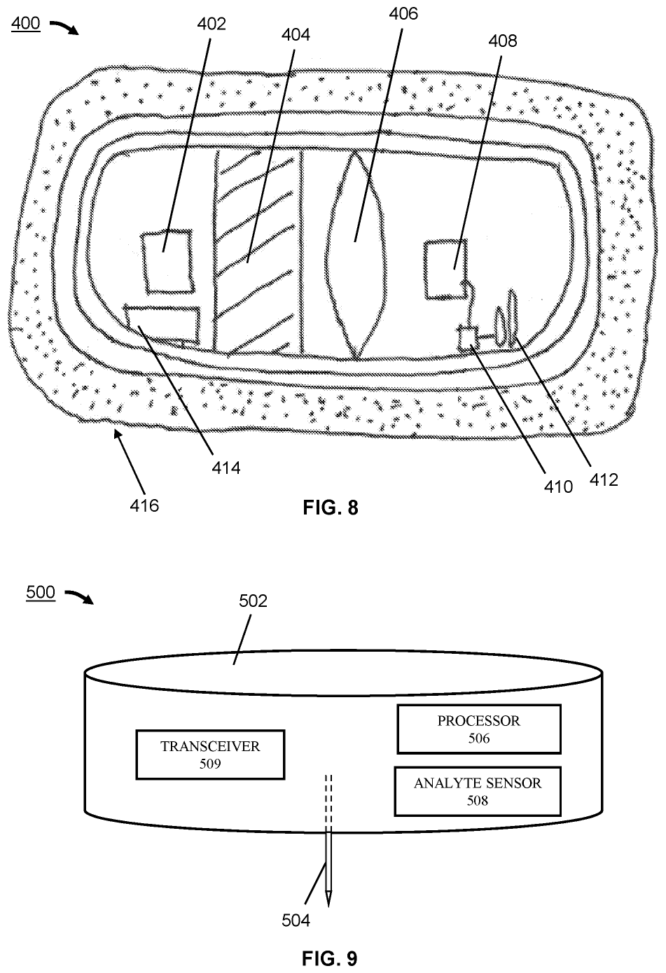

FIG. 8 illustrates an exemplary implantable analyte sensor 400, such as analyte sensor 130 of FIG. 1, for use with a medical device. Analyte sensor 400 may have dimensions of about one centimeter by one centimeter by one half centimeter.

Analyte sensor 400 may be implanted subcutaneously within a patient to calculate quantitative analyte measurements. Measurements made by analyte sensor 400 may be communicated to other devices, such as a medical device and/or mobile device. In certain embodiments, the measurements are made at continuous time intervals, which may be set and/or changed by a physician or patient via an application, as detailed above.

Analyte sensor 400 may be a fluorescence sensor which emits light with varying intensity depending on the concentration of the analyte being measured (more intense with greater concentration of analyte). An LED light source 402 may be housed within analyte sensor 400. Light from the LED 402 may pass through a matrix 404 of fluorescent indicator molecules.

In operation, as analytes come into contact with matrix 400, the fluorescent properties of the indicator material may change in proportional to the concentration of analyte. The light from the LED 402, after it has passed through the matrix 404 may be filtered through a filter 406 and directed into a photodetector 408 that detects the intensity of the light emitted. This information would be relayed to a processor 410, such as a microprocessor, which may then relay it to a transmitter 412 to, for example, wireless transmit the information to other devices. Analyte sensor 400 may include a battery 414, such as a lithium-ion battery, which may be recharged by RF wireless charging.

Analyte sensor 400 may be configured to measure one or more hormones, such as testosterone and its derivates, estrogen and its derivatives, progesterone and its derivatives, luteinizing hormone, follicle stimulating hormone, prolactin, and other substances such as hemoglobin.

Analyte sensor 400 may include a biocompatible outer layer 416. Biocompatible outer layer 416 may prevent the formation of a fibrotic capsule, which would prevent analytes from reaching the sensor 400. Biocompatible outer layer 416 may be made of made of MEDPOR Biomaterial, which may include tissue ingrowth and a cellulose membrane. The cellulose membrane may be similar to membranes used in dialysis to filter particles with the molecular weight of the analyte of interest, such as testosterone and/or hemoglobin.

Analyte sensor 400 may be calibrated based on a validated lab test for testosterone and hemoglobin. For example, analyte sensor 400 may receive a signal, such as from an external device, that the patient is having blood drawn for calibration. In response to the signal, analyte sensor 400 may measure the level of testosterone or hemoglobin. Lab values may then be compared to the values determined by sensor 400 for calibration purposes. This process may occur on a weekly, bi-weekly, month cycle. In certain embodiments, analyte sensor 400 is calibrated every three months.

FIG. 9 illustrates a non-implantable wearable device 500 configured to calculate quantitative analyte measurements. Wearable device 500 may include an adhesive patch 502 having a needle 504 extending from the underside.

Patch 502 may be made from a waterproof material and house a processor 506, an analyte sensor 508, and a transceiver 509. Transceiver 509 may transmit and receive wireless signals. The wireless signals may be Bluetooth, IEEE 802.11 wireless local area network signals, long range signals such as cellular telephone signals, near-field communications signals, or other wireless signals.

In addition, patch 502 may be worn for a 3-14 day stretch, to monitor testosterone level variations throughout the day and learn the patient's pattern of testosterone requirements by time of day. This information may then be sent to a medical device including a pump, such as medical device 100, such that testosterone replacement matching the patient's circadian cycle are administered. The patient could then wear the patch again in 1-3 months to reassess testosterone requirements and recalibrate the pump with regard to how much testosterone it needs to secrete at different times throughout the day.

Needle 504 may include two or more working electrodes, as well as a counter or reference electrode that may pierce through a patient skin and be positioned in a location containing interstitial fluid, a capillary bed, venule or arteriole. Needle 504 may have a length ranging from about one to about three centimeters, and preferably be about one and a half centimeters in length. In addition, needle 504 may have a 25 or 27 gauge diameter.

Similar to analyte sensor 400 of FIG. 8, analyte sensor 500 may be calibrated to a validated lab test for testosterone and hemoglobin. For example, analyte sensor 500 may receive a signal, such as from an external device, that the patient is having blood drawn for calibration. In response to the signal, analyte sensor 500 may measure the level of testosterone, such as through one or more of the methods described below. Lab values may then be compared to the values determined by sensor 500 for calibration purposes. This process may occur on a weekly, bi-weekly, month cycle. In certain embodiments, analyte sensor 400 is calibrated every three months.

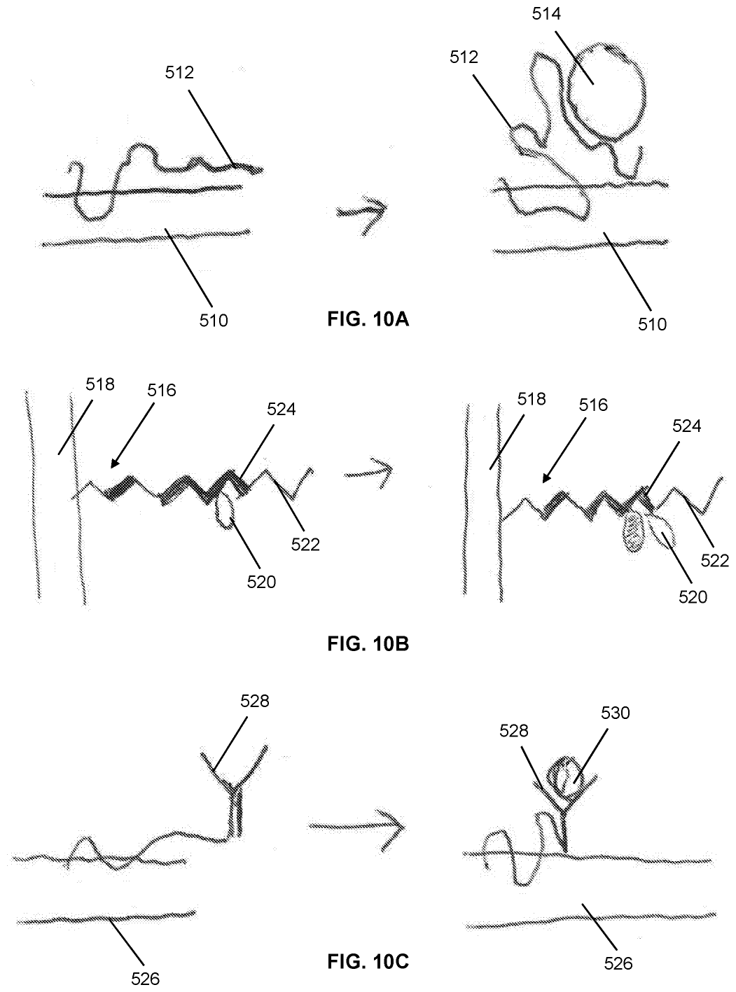

FIGS. 10A, 10B, and 10C illustrate methods for detecting levels of testosterone within a patient through use of carbon nanotube 510, such as single wall carbon nanotubes (SWCNT), attached to the ends or holes of needle 504 of non-implantable wearable device 500.

As shown in FIG. 10A, one method for detecting levels of testosterone includes the use of an aptamer 512. Aptamers, for purposes of this application, refer to a three-dimensional structure of single strand DNA or RNA, which is similar to an antigen-antibody reaction.

Aptamers configured to bind to specific target analytes may be selected, for example, by synthesizing an initial heterogeneous population of oligonucleotides, and then selecting oligonucleotides within the population that bind tightly to, for example, testosterone molecules 514. Once an aptamer that binds to a particular target molecule has been identified, it can be replicated using a variety of techniques, such as cloning and polymerase chain reaction (PCR) amplification followed by transcription.

In order to expose aptamers to a solution possibly containing target molecules, they need to be bound to a suitable substrate. To detect a binding event, it is advantageous if the substrate is conductive or semiconductive, and if it has a large specific surface area. One example of a semiconducting substrate is a carbon nanotubes.

Carbon nanotubes are allotropes of carbon with a cylindrical structure. The aptamer may be attached to the nanotubes using either covalent or non-covalent approaches. When a carbon nanotube is coated with an aptamer and then exposed to the analyte that the coated aptamer binds to, the large number of binding events and the change in conductivity of the aptamer-coated carbon nanotube will be detectable by electronic means, for example by detecting a change in conductivity, capacitance, impedance, or inductance, potentially under high-frequency alternating current. Such aptamer binding events can also affect the conductivity of metallic nanotubes.

More specifically, semiconducting SWCNT are fluorescent in the near-infrared (NIR, 900-1600 nm) due to their electronic band-gap between valence and conduction band. The semiconducting forms of SWNTs can display distinctive near-infrared (IR) photoluminescence arising from their electronic band gap. IR is a wavelength range penetrant to tissue, and thus potentially suitable for implantable sensors or other devices. The band-gap energy is sensitive to the local dielectric environment around the SWNT, and this property can be exploited in chemical sensing.

In one example, a specific interaction of the aptamer with testosterone can, for example, modulate nanotube band gap fluorescence affecting fluorescence intensity, e.g., through charge transfer, or shifting the emission wavelength(s), which can be mediated through induced dipole or bathochromic interactions. Interaction of testosterone with the aptamer may for example, increase fluorescence intensity, or decrease fluorescence intensity or the interaction may shift one or more wavelengths of fluorescence.

Analyte sensor 508 of wearable device 500 may be configured to detect fluorescence emitted by the SWNT 510 and convert the signal detected into data representing, for example, a level of testosterone within a patient. In some embodiments, a source of electromagnetic radiation may provide electromagnetic radiation of appropriate wavelength for exciting luminescence of the SWNT 510, which can be detected by the analyte sensor 508. Any known source appropriate for the sensor application can be employed including light emitting diodes, or lasers. It is noted that the excitation source may be remote from the sensor and may also be remote from the optical receptor.

FIG. 10B illustrates a method using a Corona Phase Molecular Recognition (CoPhMoRE) technique for detecting levels of testosterone within a patient. In this method, a polymer 516 may be produced with an analyte-specific binding pocket. Polymer 516 may be attached to a SWCNT 518. As detailed below, when a testosterone molecule attaches to the binding pocket, it causes the SWCNT 518 to change conformation thereby changing the near-infrared wavelength it emits.

In terms of creating a polymer that is specific to testosterone, a library of polymers could be created using RAFT polymerization to create random copolymers. Random copolymers may include a hydrophilic unit 522, which gives colloidal stability at physiologic pH, such as acrylic acid. Furthermore, random copolymers may include a hydrophobic unit 524 of the polymer, which attaches to the SWCNT. In certain embodiments, the hydrophobic unit 524 may be styrene and acrylated testosterone may serve as a template for the analyte binding pocket of the polymer. These three elements help create the “corona” of the polymer, which necessarily excludes molecules other than testosterone from interacting with the SWCNT.

As shown in FIG. 10B, acrylated testosterone 520 may be an appendage and/or displaced off the polymer backbone. The acrylated testosterone 520 may be adsorbed and desorbed from the cavity created by the backbone, which leaves a high-fidelity, reversible binding pocket for testosterone. From the library created, the polymer that provides the best specificity for testosterone could be selected.

In certain embodiments, the SWCNT 518 can be encapsulated in a hydrogel. Emission from the SWCNT 518 can be assayed using analyte sensor 508, which may include a fiber optic probe-based system with a laser to excite the SWCNTs. Emission from the SWCNT can be collected through the same fiber bundle, which is coupled to a spectrometer/NIR array detector. This information is then relayed to the processor 506 of the patch 502 to, for example, determine the concentration of testosterone based on the wavelength shift detected.

FIG. 10C illustrates another method for detecting levels of testosterone with a patient. As shown, a SWCNT 526 may be complexed with an anti-testosterone antibody 528 (i.e., an antibody specific for testosterone).

In the illustrated method of FIG. 10C may be performed without chemical perturbation of the graphitic carbon of the nanotube 526. This may be created by suspending SWCNTs with single-stranded DNA oligonucleotides (for example, 6 repeats of TAT nucleotides) modified at the 3′ end of the oligonucleotide with a primary amine functional group, via ultrasonication. The suspension may then be purified by ultracentrifugation to remove bundles. Excess DNA may be removed by centrifugal filtration. The DNA-SWCNT complex may then be conjugated via carbodiimide cross-linker chemistry to a polyclonal anti-testosterone IgG antibody (Ab), dialyzed against water for 48 hours to remove unreacted agents.

The Ab-DNA-SWCNT may then be loaded into a semipermeable membrane, such as a polyvinylidene fluoride (PVDF) membrane capillary, which may serve as a sieve. The sieve may have a molecular weight cutoff that is larger than that of testosterone (to allow the testosterone molecules in), but smaller than the molecular weight of the Ab-DNA-SWCNT complex (to keep the complexes from going out). When a testosterone molecule 530 binds the Ab-DNA-SWCNT complex, the wavelength of the near-infrared (NIR) emitted by the Ab-DNA-SWCNT complex shifts. The emission may be analyzed by analyte sensor 508, such as through use of the fiber optic probe-based system described above.

In certain embodiments, analyte sensor 508 of wearable device 500 may detect testosterone levels every 10 seconds using one of the above illustrated methods, and average the values obtained in a ten-minute period. The information may then be transmitted to, for example, a medical device and/or a mobile device.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described in the application are to be taken as examples of embodiments. Components may be substituted for those illustrated and described in the application, parts and processes may be reversed, and certain features of the invention may be utilized independently, all as would be apparent to one skilled in the art after having the benefit of this description of the invention. Changes may be made in the elements described in the application without departing from the spirit and scope of the invention as described in the following claims.

What is claimed is:

1. An implantable medical device for delivering testosterone, the device comprising:
a front surface including one or more openings for one or more channels;
at least one reservoir connected to said one or more channels, each reservoir configured to hold testosterone;
a catheter extending from a side surface and connected to said at least one reservoir, said catheter for delivering said testosterone to a patient; and
a pump connected to said catheter, said pump configured to dynamically regulate the delivery of said testosterone.

2. The medical device of claim 1, wherein each opening includes a pierceable and reclosable membrane.

3. The medical device of claim 1, wherein said one or more channels include a detector configured to detect the presence of a needle for refilling said at least one reservoir.

4. The medical device of claim 1, wherein each reservoir includes a sensor configured to measure an amount of testosterone corresponding to the reservoir.

5. The medical device of claim 1, wherein said front surface further including a plurality of light emitting diodes (LEDs) positioned around each opening, said LEDs configured to illuminate in response to at least one of a refilling operation and a notification.

6. The medical device of claim 1, wherein said pump is operatively coupled to a continuous analyte sensor configured to measure a testosterone level of the patient.

7. The medical device of claim 6, wherein said analyte sensor extends from a side surface opposite said catheter assembly.

8. The medical device of claim 6, wherein said analyte sensor is remotely implanted subcutaneously within the patient.

9. The medical device of claim 1, further comprising a transceiver configured to receive a control signal from one or more external devices.

10. The medical device of claim 9, wherein said pump is configured to increase, decrease or stop the delivery of said testosterone in response to a control signal from the one or more external devices.

11. The medical device of claim 1, wherein the testosterone is selected from a group consisting of unmodified testosterone, testosterone propionate, testosterone enanthate, testosterone undecanoate, testosterone cypionate, testosterone undecylenate, other testosterone derivates, human chorionic growth hormone, conjugated estrogens, estradiol, esterified estrogens, progesterone, methylprogesterone, progesterone derivates, and anastrazole.

12. The medical device of claim 1, further comprising a speaker configured to output audible notifications.

13. A wearable patch comprising:
an analyte sensor;
a transceiver;
a processor, operatively coupled to the analyte sensor and to the transceiver, the processor operative to:
obtain testosterone levels collected using the analyte sensor; and
send the testosterone levels obtained using the analyte sensor to a second device over a wireless link using the transceiver.

14. The wearable patch of claim 13, further comprising a needle including one or more carbon nanotubes, wherein the carbon nanotubes are single-walled carbon nanotubes or multi-walled carbon nanotubes.

15. The wearable patch of claim 13, wherein analyte sensor is configured to collect the testosterone levels using at least one of a corona phase molecular recognition technique, an anti-testosterone antibody, and an aptamer specific for testosterone.

16. A system for dynamically controlling delivery of testosterone comprising the wearable patch of claim 13, and further comprising:
an implantable medical device including a pump, wherein the medical device is the second device, the medical device comprising:
a transceiver;
a processor operatively coupled to the transceiver;
a non-volatile, non-transitory memory storing one or more modules with instruction executed by the processor, the processor operative to:
analyze the testosterone levels collected by the analyte sensor;
determine a flow rate for dispensing testosterone; and
output a control signal to the pump, the control signal configured to cause the pump to deliver the testosterone to a patient at the determined flow rate.

17. The system of claim 16, wherein the processor is further operative to:
obtain the flow rate from a lookup table stored in the non-volatile, non-transitory memory comprising a plurality of entries, each entry corresponding to the testosterone.

18. The system of claim 16, wherein the processor is further operative to:
receive, from one or more external devices, the flow rate; and
adjust the control signal, thereby changing the flow rate such that the pumping mechanism is configured to increase, decrease or stop the delivery of said one or more testosterone.

19. The system of claim 16, wherein the medical device further comprises one or more sensors operatively coupled to the processor.

20. The system of claim 19, wherein the processor is further operative to:
analyze information collected by the one or more sensors; and
communicate, in response to the information, one or more notifications to the patient.
